(19) United States
(12) Patent Application Publication
(10) Pub. No.: US 2024/0242814 A1
(43) Pub. Date: Jul. 18, 2024

(54) INFORMATION PROCESSING DEVICE, INFORMATION PROCESSING METHOD, AND PROGRAM

(71) Applicant: CYKINSO, INC., Tokyo (JP)

(72) Inventors: Yu SAWAI, Tokyo (JP); Satoshi WATANABE, Tokyo (JP); Takayoshi FUKUSHIMA, Tokyo (JP); Yusuke MATSUO, Tokyo (JP)

(73) Assignee: CYKINSO, INC., Tokyo (JP)

(21) Appl. No.: 18/573,708

(22) PCT Filed: Mar. 23, 2022
(86) PCT No.: PCT/JP2022/013685

§ 371 Date: Mar. 12, 2024

(30) Foreign Application Priority Data
Jun. 25, 2021 (JP) 2021-105767

(51) Int. Cl. G16H 20/60

(52) U.S. Cl. CPC G16H 20/60

(57) ABSTRACT

The present invention addresses the problem of providing a method that enables more accurate suggestions for improving both the health and beauty conditions of a user. A second acquisition unit 121 acquires the user's gut bacteria data. A score determination unit 122 determines a score that indicates the health level of the gut microbiome of the user on the basis of the gut bacteria data. A type determination unit 123 determines the type to which the user's gut flora applies, among a plurality of enterotypes, on the basis of the gut bacteria data. An information unit 125 informs suggestions for improving the user's health condition to the user on the basis of the user's score and type. The problem is solved thereby.

### TECHNICAL FIELD

The present invention relates to an information processing device, an information processing method, and a program.

### BACKGROUND ART

In order to grasp a health condition of a subject, human indigenous bacteria of the subject have been conventionally inspected (for example, see Patent Document 1). The above-described technique disclosed in Patent Document 1 supports a diet necessary to achieve preferable human indigenous bacteria on the basis of the correlation between preset human indigenous bacteria and diet, for example.

### CITATION LIST

### Patent Document

Patent Document 1: Japanese Unexamined Patent Application, Publication No. 2012-165716

### DISCLOSURE OF THE INVENTION

### Problems to be Solved by the Invention

However, there has been a demand for development of a method that enables more accurate suggestions for improving both health and beauty conditions of a subject than conventional techniques including the above-described technique disclosed in Patent Document 1. The present invention has been made in view of such circumstances, and an object thereof is to provide a method that enables more accurate suggestions for improving both health and beauty conditions of a subject.

### Means for Solving the Problems

In order to achieve the above-described object, an information processing device of one aspect of the present invention comprises:
acquisition unit that acquires gut bacteria data of a user; first determination unit that determines a score that indicates a health level of a gut microbiome of the user on a basis of the gut bacteria data;
second determination unit that determines a type to which gut microbiota of the user applies, among a plurality of enterotypes on the basis of the gut bacteria data; and information unit that informs suggestions for improving a health condition of the user to the user on the basis of the score and the type of the user.

An information processing method and a program that correspond to the above-described information processing device of one aspect of the present invention are provided as an information processing method and a program of one aspect of the present invention.

### Effects of the Invention

According to the present invention, more accurate suggestions for improving both health and beauty conditions of a user are enabled.

### BRIEF DESCRIPTION OF THE DRAWINGS

FIG. 1 is a diagram illustrating an overview of a present service that can be implemented by an information processing system to which a server according to one embodiment of the present invention is applied;

FIG. 2 is a diagram illustrating a specific example of enterotypes;

FIG. 3 is a diagram illustrating a specific example of groupings of subjects;

FIG. 4 is a diagram illustrating one example of a configuration of an information processing system to which a server according to one embodiment of the information processing device of the present invention is applied;

FIG. 5 is a block diagram illustrating one example of a hardware configuration of the server in the information processing system illustrated in FIG. 4;

FIG. 6 is a functional block diagram illustrating one example of a functional configuration for executing grouping processing and recommendation processing, in the functional configuration of the server in FIG. 5 constituting the information processing system in FIG. 4;

FIG. 7 is a flowchart illustrating the flow of the grouping processing and the recommendation processing by the server having the functional configuration in FIG. 6;

FIG. 8 is a diagram illustrating a specific example of a screen displayed on a user terminal when a gut dysbiosis score is informed to a subject;

FIG. 9 is a diagram illustrating a specific example of the screen displayed on the user terminal when an enterotype is informed to the subject;

FIG. 10 is a diagram illustrating a specific example of the screen displayed on the user terminal when the subject answers a questionnaire; and

FIG. 11 is a diagram illustrating a specific example of the screen displayed on the user terminal when dietary supplements are recommended to the subject.

### PREFERRED MODE FOR CARRYING OUT THE INVENTION

Hereinafter, embodiments of the present invention will be described with reference to the drawings.

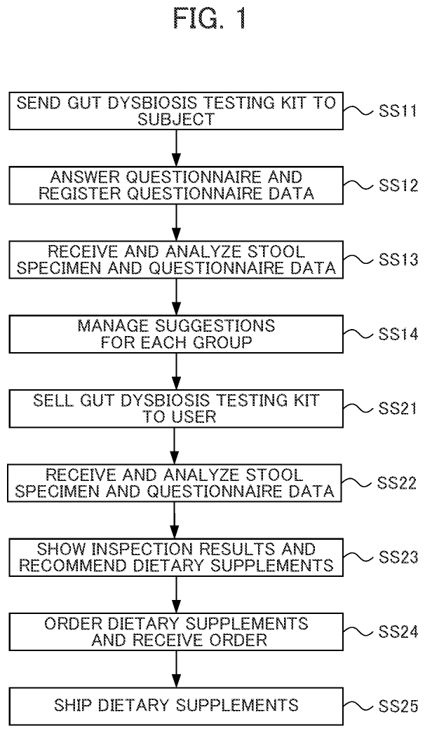

First, referring to FIGS. 1 to 3, a description will be given with respect to an overview of service (hereinafter, referred to as the “present service”) that can be implemented by an information processing system to which a server 1 according to one embodiment of the present invention is applied.

FIG. 1 is a diagram illustrating an overview of the present service that can be implemented by an information processing system to which a server according to one embodiment of the present invention is applied.

The present service is service that is provided by a service provider (not illustrated) and provides suggestions on health and beauty tailored to a user on the basis of a score and type of gut microbiota of the user. Specifically, in the present service, first, the analyses are performed on a stool specimen and questionnaire on a health condition that are provided from each of n (n is an integer value of two or more) subjects. Then, a score (hereinafter, referred to as an “gut dysbiosis score”) that indicates whether the gut microbiome is good or bad and a type of gut microbiota (hereinafter, referred to as an “enterotype”) are determined on the basis of the results of the analyses. The subjects are grouped on the basis of a combination of these determination results, and appropriate suggestions on the beauty and health are managed for each group. These suggestions include, for example, recommendations on brands of dietary supplements (hereinafter, may be simply referred to as “supplements”) effective for improving the beauty and health of a subject, among a plurality of kinds of dietary supplements commercially available. Additionally, suggestions are provided on nutrients, foods, medications, and physical exercise that are necessary to maintain and improve the beauty and health. Specifically, a stool specimen of a user of the present service is analyzed, a determination is made as to which group the user belongs to, and suggestions managed corresponding to the group to which the user belongs are provided. Note that the user may be a person other than the above-described n subjects or any one of the n subjects.

The present service is provided in the flow illustrated in FIG. 1. That is, a gut flora testing kit is sent to a subject from a service provider (service step SS11). The “gut flora testing kit” refers to a testing kit for determining a gut dysbiosis score of the subject and determining an enterotype. The gut flora testing kit includes an instrument for collecting a stool specimen by a subject himself or herself to send the stool specimen to the service provider, and a questionnaire on a health condition to be answered by the subject.

The subject answers the questionnaire on the health condition. Then, the subject sends the questionnaire together with the subject's stool specimen collected by himself or herself to a destination specified by the service provider (service step SS12). The questionnaire includes, for example, an item in which troubles on the health of the subject can be entered. The questionnaire is not limited to a particular form. Examples of the form to be filled in may include a paper-based form. In the present embodiment, a form is adopted in which the subject accesses a predetermined website specified by the service provider and fills in a predetermined input form. When the subject inputs the questionnaire answer to the predetermined input form, the input contents are registered as data on the health condition.

Here, the data on the health condition may include any data on the health in addition to the above-described questionnaire data. Specifically, for example, data that can be acquired in cooperation with service different from the present service (for example, exercise amount management service, medical check-up result browsing service, and medication notebook service) is one example of the above-described data on the health condition.

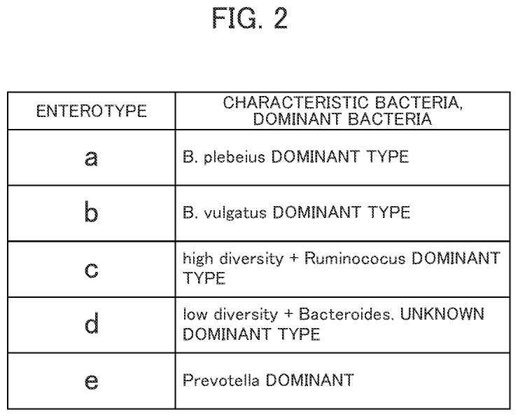

The stool specimen and the registered data on the health condition that are sent from the subject are received by the service provider and are managed for each subject. Then, various analyses on these are performed (service step SS13). Specifically, the analyses are performed to determine a gut dysbiosis score of the subject and determine an enterotype. Note that a specific example of the enterotype will be described later with reference to FIG. 2.

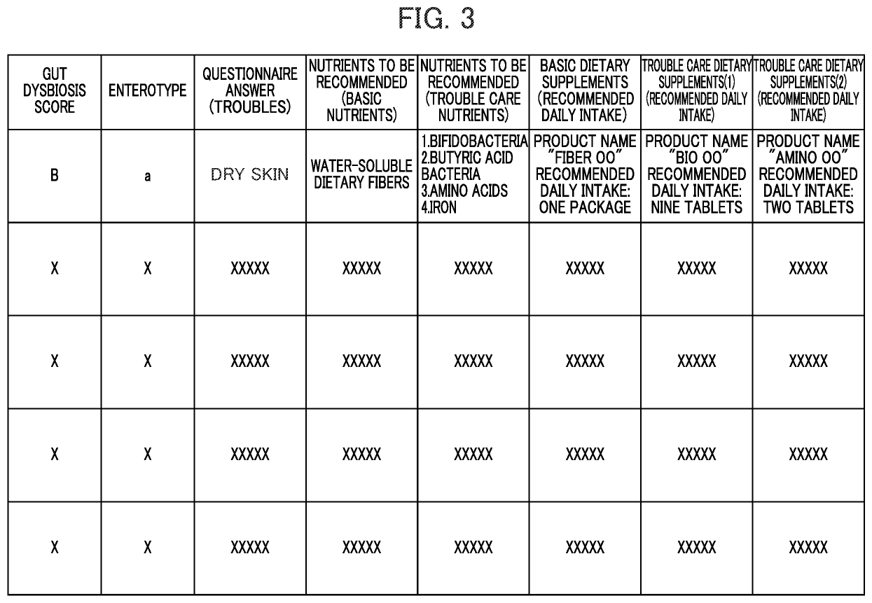

That is, the service provider performs grouping to classify each of the n subjects into any one of a plurality of groups on the basis of the analysis results of the stool specimen and data on the health condition for each of the n subjects. At this time, the recommendations of dietary supplements are managed in a group unit (service step SS14). Here, the method of grouping is not limited to a particular method. Since the same dietary supplements are recommended for subjects belonging to the same group, it is preferable that the subjects are classified into a group of subjects with similar gut dysbiosis scores, a group of subjects with the same enterotype, a group of subjects with similar troubles on the health input in the questionnaires, and the like, for example. Note that a specific example of classification into groups will be described later with reference to FIG. 3.

The data on the analysis results of the stool specimen of the subject or user is managed as “gut bacteria data”. The “gut bacteria data” is data including at least information about two or more types of gut microbiome. For example, regarding a plurality of enterotypes collected from the stool specimen, data that indicates an amount of each of the plurality of enterotypes present in the stool specimen is one example of the gut bacteria data.

As described above, the service provider groups n subjects on the basis of the score and type of gut microbiota, and manages suggestions on the health and beauty tailored to each of the groups. Next, as described below, the service provider provides suggestions on the health and beauty tailored to the group to which a user belongs, on the basis of the score and type of the gut microbiota of the user.

That is, the service provider sells a gut flora testing kit to the user (service step SS21). The gut flora testing kit includes an instrument for collecting a stool specimen by the user himself or herself to send the stool specimen to the service provider, and a questionnaire on a health condition to be answered by the user. It should be noted that the contents of the questionnaire are not necessarily the same as those of the above-described questionnaire in service step SS11.

The user answers the questionnaire on the health condition. Then, the user sends the questionnaire together with the user's own stool specimen collected by himself or herself to a destination specified by the service provider (service step SS22).

The stool specimen and the registered data on the health condition that are sent from the user are received by the service provider and are managed for each user. Then, various analyses on these are performed (service step SS23). Specifically, the analyses are performed to determine a gut dysbiosis score of the user and determine an enterotype.

The service provider shows inspection results to each of the subjects on the basis of the analysis results of the stool specimen and data on the health condition of the user. The intake and purchase of dietary supplements are recommended in a group unit on the basis of which group the user belongs to among a plurality of groups (service step S23).

In the case where the subject having received the recommendations of the dietary supplements wishes to purchase the dietary supplements of interest, the subject can order the dietary supplements directly from the present service. The service provider receives the order (service step SS24), and ships the dietary supplements (service step SS225). This enables smooth implementation of service steps of recommending, ordering, receiving, and shipping the dietary supplements, the dietary supplements being appropriate to improving the health and beauty of the subject.

FIG. 2 is a diagram illustrating a specific example of enterotypes.

As illustrated in FIG. 2, in the present embodiment, the enterotypes are classified into five types of “a” to “e” on the basis of characteristic bacteria and dominant bacteria. That is, the enterotype a is a type of gut microbiota in which the characteristic bacteria and the dominant bacteria are of “B. plebeius dominant type”. The enterotype b is a type of gut microbiota in which the characteristic bacteria and the dominant bacteria are of “B. vulgatus dominant type”. The enterotype c is a type of gut microbiota in which the characteristic bacteria and the dominant bacteria are of “high diversity+Ruminococcus dominant type”. The enterotype d is a type of gut microbiota in which the characteristic bacteria and the dominant bacteria are of “low diversity+Bacteroides. unknown dominant type”. The enterotype e is a type of gut microbiota in which the characteristic bacteria and the dominant bacteria are of “Prevotella dominant type”.

FIG. 3 is a diagram illustrating a specific example of groupings of subjects.

The grouping of each of the n subjects is performed on the basis of the results of analyses the stool specimens and data on the health condition by the service provider. The grouping is performed in consideration of items including gut dysbiosis score, enterotype, questionnaire answer (troubles), nutrients (basic nutrients) to be recommended, nutrients (trouble care nutrients) to be recommended, basic dietary supplements (recommended daily intake), trouble care dietary supplements (1) (recommended daily intake), and trouble care dietary supplements (2) (recommended daily intake) as illustrated in FIG. 3. Specifically, for example, the grouping is performed in consideration of the items including the gut dysbiosis score which is “B”, the enterotype which is “a”, the questionnaire answer (troubles) which is “dry skin”, the nutrients (basic nutrients) to be recommended which is “water-soluble dietary fibers”, the nutrients (trouble care nutrients) to be recommended which is “1. Bifidobacteria, 2. Butyric acid bacteria, 3. Amino acids, 4. Iron”, the basic dietary supplements (recommended daily intake) which is “Product name “Fiber OO”, Recommended daily intake: one package”, the trouble care dietary supplements (1) (recommended daily intake) which is “Product name “Bio OO”, Recommended daily intake: nine packages”, and the trouble care dietary supplements (2) (recommended daily intake) which is “Product name “Amino OO”, Recommended daily intake: two tablets”. That is, as illustrated in FIG. 3, the grouping and recommendations are performed on the basis of the gut dysbiosis score, the enterotype, and the data on the health (in this example, the questionnaire answer (troubles)).

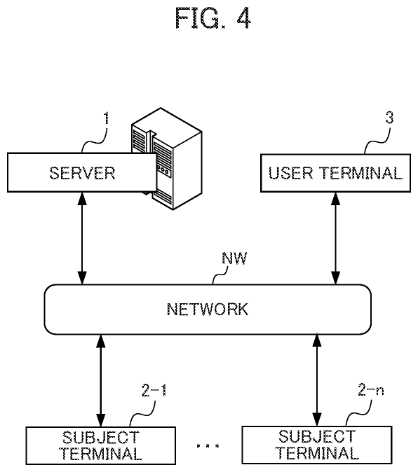

Next, referring to FIG. 4, a description will be given with respect to a configuration of an information processing system that achieves provision of the above-described present service, that is, an information processing system to which the server 1 according to one embodiment of the information processing device of the present invention is applied. FIG. 4 is a diagram illustrating one example of the configuration of the information processing system to which a server according to one embodiment of the information processing device of the present invention is applied.

The information processing system illustrated in FIG. 4 is configured to include the server 1, subject terminals 2-1 to 2-n, and a user terminal 3. The server 1, the subject terminals 2-1 to 2-n, and the user terminal 3 are connected with each other through a predetermined network NW such as the Internet.

The server 1 is an information processing device managed by the service provider. The server 1 executes various types of processing for implementing the present service while appropriately communicating with the subject terminals 2-1 to 2-n. When it is not necessary to discriminate among the individual subject terminals 2-1 to 2-n, they are collectively referred to hereinafter as a “subject terminal 2”.

The subject terminal 2 is an information processing device operated by a subject. The subject terminal 2 is configured with a personal computer, a tablet, a smartphone, or the like. The subject terminal 2 receives input operations of various types of information by the subject to transmit the information to the server 1, and receives various types of information transmitted from the server 1 to display the information thereon.

The user terminal 3 is an information processing device operated by a predetermined user. Specifically, for example, the user terminal 3 is operated by a user other than the n subjects or any one of the n subjects. The user terminal 3 is configured with a personal computer, a tablet, a smartphone, or the like. The user terminal 3 receives input operations of various types of information by the user to transmit the information to the server 1, and receives various types of information transmitted from the server 1 to display the information thereon.

The subject terminal 2 and the user terminal 3 can have dedicated application software installed therein, the software allowing comfortable use of the present service. Even when the dedicated application software is not installed, the present service can be used also by accessing the dedicated website allowing use of the present service.

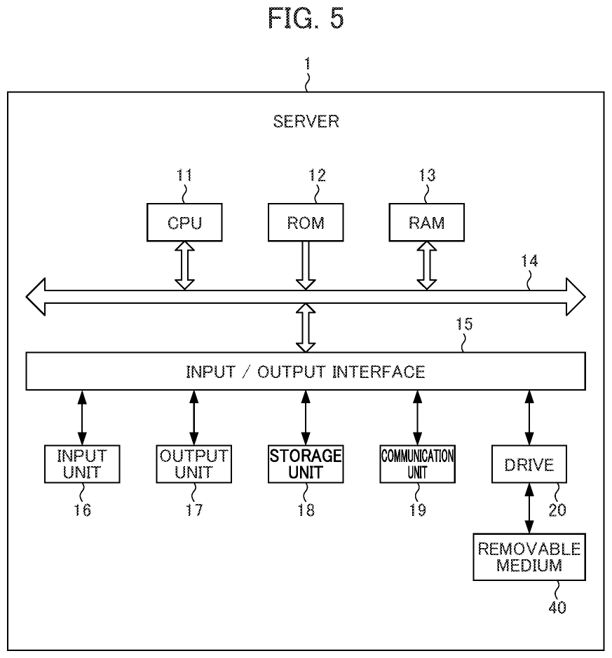

FIG. 5 is a block diagram illustrating one example of a hardware configuration of the server in the information processing system illustrated in FIG. 4.

The server 1 comprises a central processing unit (CPU) 11, a read only memory (ROM) 12, a random access memory (RAM) 13, a bus 14, an input/output interface 15, an input unit 16, an output unit 17, a storage unit 18, a communication unit 19, and a drive 20.

The CPU 11 executes various types of processing according to programs recorded in the ROM 12 or programs loaded from the storage unit 18 to the RAM 13. In the RAM 13, data that is necessary for the CPU 11 to execute various types of processing is also appropriately stored.

The CPU 11, the ROM 12, and the RAM 13 are connected with each other via the bus 14. The input/output interface 15 is also connected to the bus 14. The input unit 16, the output unit 17, the storage unit 18, the communication unit 19, and the drive 20 are connected to the input/output interface 15.

The input unit 16 is configured with, for example, a keyboard, or the like, and is used to input various types of information. The output unit 17 is configured with a display of liquid crystal or the like, a speaker, or the like, and outputs various types of information as images or sounds. The storage unit 18 is configured with a dynamic random access memory (DRAM) or the like, and stores various types of data. The communication unit 19 communicates with other devices (for example, the subject terminal 2 in FIG. 4) through the network NW including the Internet.

A removable medium 40 including a magnetic disk, an optical disc, a magneto-optical disk, a semiconductor memory, or the like is appropriately mounted on the drive 20. A program read from the removable medium 40 by the drive 20 is installed in the storage unit 18 if necessary. The removable medium 40 can also store various types of data stored in the storage unit 18, similarly to the storage unit 18.

Although not illustrated, the subject terminal 2 and the user terminal 3 in FIG. 4 can also have basically the same configuration as that of the hardware configuration illustrated in FIG. 5. Therefore, the description of the hardware configuration of the subject terminal 2 is omitted.

Such cooperation between various types of hardware constituting the information processing system in FIG. 4 including the server 1 in FIG. 5 and various types of software allows execution of various types of processing including the grouping processing and the recommendation processing. As a result, the service provider can provide the above-described present service to the subjects. The “grouping processing” refers to processing that is executed to classify each of n subjects into a plurality of groups on the basis of the gut bacteria data and the data on the health condition of each of the n subjects. The “recommendation processing” refers to processing that is executed to recommend dietary supplements corresponding to the group to which a user belongs, on the basis of the gut bacteria data and the data on the health condition of the user. In this way, as a result of the recommendation processing, the same kinds of dietary supplements are recommended to a plurality of users belonging to the same group.

In the grouping processing and the recommendation processing, the gut dysbiosis score and the enterotype are determined on the basis of the above-described gut bacteria data, and appropriate suggestions on the beauty and health are managed for each group of the subjects based on a combination of the determination results. At this time, the data on the health condition is used for the grouping processing, the recommendation processing, and generation of the contents (suggestions) of the recommendations.

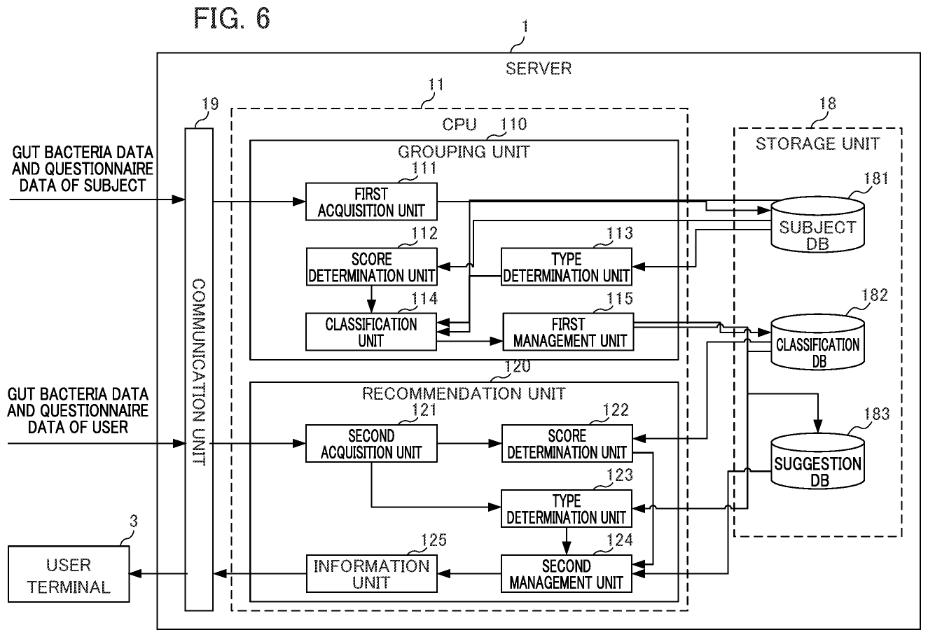

Here, the advantage of recommending the same kinds of dietary supplements in a group unit will be described. That is, since the health condition, the physical condition, a way of thinking about the health, troubles about the health, and the like are common among one or more users belonging to the same group, the types of dietary supplements effective for improving the beauty and health of a subject are also common among the users. When the service provider recommends new dietary supplements to the users, it is more efficient to examine the new dietary supplements to be recommended to which subjects in a group unit rather than to examine individually. The dietary supplements determined as being highly effective in improving the beauty of a certain user can be expected to show the same effect also for other users belonging to the same group as the user. This enables targeted recommendations. Hereinafter, referring to FIG. 6, a description will be given with respect to a functional configuration for executing the grouping processing and the recommendation processing that are executed in the server 1 in FIG. 5 constituting the information processing system in FIG. 4.

FIG. 6 is a functional block diagram illustrating one example of a functional configuration for executing the recommendation processing, in the functional configuration of the server in FIG. 5 constituting the information processing system in FIG. 4.

As illustrated in FIG. 6, in the case where the server 1 executes the grouping processing, in the CPU 11, a recommendation unit 110 functions. The recommendation unit 110 includes a first acquisition unit 111, a score determination unit 112, a type determination unit 113, a classification unit 114, and a first management unit 115.

In one region of the storage unit 18 in the server 1, a subject DB 181, a classification DB 182, and a suggestion DB 183 are provided. In the subject DB 181, the gut bacteria data and the data on the health condition are managed in association with the information (for example, ID) capable of uniquely identifying the subject. In the classification DB 182, the information about the groups into which the n subjects are classified is managed. In the suggestion DB 183, a group and the information about the dietary supplements to be recommended to the group is managed in association with each other for each group into which the n subjects are classified.

The first acquisition unit 111 acquires, as the gut bacteria data, data including at least the information about gut bacteria of each of the n subjects. The first acquisition unit 111 acquires, as the data on the health condition, data including at least the information about the health condition of each of the n subjects. The gut bacteria data and the data on the health condition that are acquired by the first acquisition unit 111 are stored in and managed by the subject DB 181.

The score determination unit 112 determines a gut dysbiosis score as a score that indicates the health level of the gut microbiome of each of the n subjects on the basis of the gut bacteria data stored in the subject DB 181.

The type determination unit 113 determines the type to which the gut microbiota of each of the n subjects applies, among a plurality of enterotypes on the basis of the gut bacteria data stored in the subject DB 181.

The classification unit 114 classifies each of the n subjects into any one of a plurality of groups on the basis of the gut dysbiosis score, the enterotype, and the data on the health condition of each of the n subjects.

The first management unit 115 manages suggestions for improving the health conditions of persons belonging to the same group for each of the groups classified by the classification unit 114. Specifically, for example, the first management unit 115 stores in the classification DB182 and manages the information about groups into which the n subjects are classified by the classification unit 114. For example, the first management unit 115 stores in the classification DB 182 and manages the information as to gut bacteria data and data on the health condition which are available to the users belonging to each group. The first management unit 115 stores in the suggestion DB 183 and manages a group in association with the information about the dietary supplements to be recommended to the group. For example, the first management unit 115 stores in the classification DB 182 and manages, for each group, the information about the dietary supplements to be recommended to persons belonging to the group.

The n subjects are grouped by the server 1 having the above-described functional configuration. Next, the functional configuration of the server 1 for executing the recommendation processing will be described.

As illustrated in FIG. 6, in the case where the server 1 executes the recommendation processing, in the CPU 11, a recommendation unit 120 functions. The recommendation unit 120 includes a second acquisition unit 121, a score determination unit 122, a type determination unit 123, a second management unit 124, and an information unit 125.

The second acquisition unit 121 acquires, as the gut bacteria data, data including at least the information about gut bacteria of a user to be subjected to recommendations. The second acquisition unit 121 acquires, as the data on the health condition, data including at least the information about the health condition of the user.

The score determination unit 122 determines a gut dysbiosis score as a score that indicates the health level of the gut microbiome of the user on the basis of the gut bacteria data of the user to be subjected to recommendations that is acquired by the second acquisition unit 121.

The type determination unit 123 determines the type to which the gut microbiota of the user applies, among a plurality of enterotypes on the basis of the gut bacteria data of the user to be subjected to recommendations that is acquired by the second acquisition unit 121.

The second management unit 124 manages the gut dysbiosis score determined by the score determination unit 122 and suggestions to the group based on the enterotype determined by the type determination unit 123. Specifically, the second management unit 124 manages the group to which the user belongs, on the basis of the group based on the gut dysbiosis score and the enterotype of the user and the information about the groups stored in the classification DB 182. Then, the second management unit 124 acquires the information about the dietary supplements to be recommended to a user on the basis of the group to which the user belongs and the information about the dietary supplements to be recommended that are stored in the suggestion DB 183. The second management unit 124 can further acquire the information about the dietary supplements to be recommended to a user on the basis of the information about the health of the user (in the example in FIG. 3, the questionnaire answer (troubles)).

The information unit 125 informs, to a user to be subjected to recommendations, the suggestions preset to the group to which the user belongs, among the suggestions for improving the health condition preset for each group. That is, the information unit 125 informs, to a user to be subjected to recommendations, the suggestions (suggestions managed in association with the group in the suggestion DB 183) present to the group to which the user belongs, the group being managed by the second management unit 124, on the basis of the gut bacteria data and the data on the health condition of the user to be subjected to recommendations that are acquired by the second acquisition unit 121. Specifically, for example, the information unit 125 suggests intake and purchase of the same kinds of dietary supplements as suggestions for improving the health condition of the user to be subjected to recommendations.

Specifically, for example, the second management unit 124 determines which group the user to be subjected to recommendations belongs to, among the groups managed using the classification DB 182, on the basis of the gut bacteria data and the data on the health condition that are acquired by the second acquisition unit 121. The information unit 125 acquires, from the suggestion DB 183, the suggestions preset to the group corresponding to the group to which the user to be subjected to recommendations belongs, the group being determined by the second management unit 124, and informs the acquired suggestions to the user. Although not illustrated, the first management unit 115 may determine the group to which the user belongs to, using the determination result of the gut dysbiosis score of the user and the determination result of the enterotype of the user.

As described above, the information processing device in FIG. 5 has the above-described functional configuration illustrated in FIG. 6, thereby enabling more accurate suggestions for improving both health and beauty conditions of a subject.

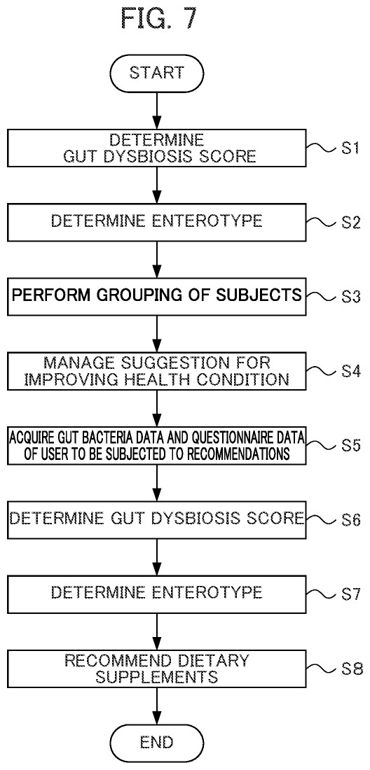

FIG. 7 is a flowchart illustrating a series of flows of the grouping processing and the recommendation processing by the server having the functional configuration in FIG. 6.

In the case where the execution of a series of processing including the grouping processing and the recommendation processing is controlled by the CPU 11 in the server 1 having the functional configuration in FIG. 6, in step S1, the score determination unit 112 determines a gut dysbiosis score of each of the n subjects on the basis of the gut bacteria data. Next in step S2, the type determination unit 113 determines an enterotype of each of the n subjects on the basis of the gut bacteria data. Next, in step S3, the classification unit 114 performs grouping to classify each of the n subjects into any one of a plurality of groups on the basis of the gut dysbiosis score, the enterotype, and the data on the health condition of each of the n subjects. Next, in step S4, the first management unit 115 manages suggestions for improving the health conditions of persons belonging to the same group for each of the groups classified by processing in step S3. Next, in step S5, the second acquisition unit 121 acquires, as the gut bacteria data, data including at least the information about gut bacteria of a user to be subjected to recommendations. The second acquisition unit 121 acquires, as the data on the health condition, data including at least the information about the health condition of the user. Next, in step S6, the score determination unit 122 determines a gut dysbiosis score as a score that indicates the health level of the gut microbiome of the user on the basis of the gut bacteria data and the data on the health condition of the user to be subjected to recommendations that are acquired as a result of the processing in step S5. Next, in step S7, the type determination unit 123 determines the type to which the gut microbiota of the user applies, among a plurality of enterotypes on the basis of the gut bacteria data of the user to be subjected to recommendations that is acquired as a result of the processing in step S5. Next, in step S8, the information unit 125 informs, to a user to be subjected to recommendations, the suggestions preset to the group to which the user belongs, among the suggestions for improving the health condition preset for each group, on the basis of the gut dysbiosis score and the enterotype of the user.

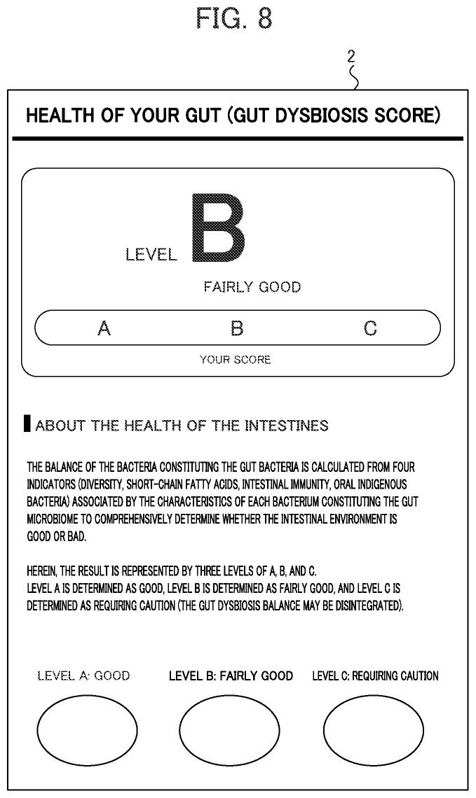

Next, a specific example of a screen displayed on the user terminal 3 will be described with reference to FIGS. 8 to 11. FIG. 8 is a diagram illustrating a specific example of a screen displayed on a user terminal when a gut dysbiosis score is informed to a user.

On the screen in FIG. 8, “Health of your gut (gut dysbiosis score)” and “Level B: Fairly good” are displayed. This indicates that the gut dysbiosis score of the user is “B” among “A” to “C”, and indicates “fairly good”. On the screen in FIG. 8, “About the health of the intestines” and the explanation about the gut dysbiosis score are displayed. Specifically, for example, as illustrated in FIG. 8, the explanation shows that “the balance of the bacteria constituting the gut bacteria is calculated from four indicators (diversity, short-chain fatty acids, intestinal immunity, oral indigenous bacteria) associated by the characteristics of each bacterium constituting the gut microbiome to comprehensively determine whether the gut microbiome is good or bad. Herein, the result is represented by three levels of A, B, and C. Level A is determined as good, Level B is determined as fairly good, and Level C is determined as requiring caution (the gut flora balance may have collapsed). Level A: good, Level B: fairly good, Level C: requiring caution”. The user can grasp easily a user's own gut dysbiosis score only by visually recognizing the screen in FIG. 8 that is displayed on the user terminal 3.

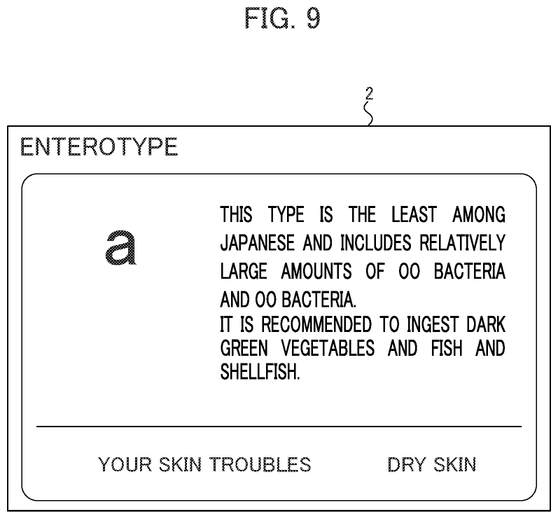

FIG. 9 is a diagram illustrating a specific example of the screen displayed on the user terminal when an enterotype is informed to the user.

On the screen in FIG. 9, “enterotype”, “a”, and the explanation are displayed. This indicates that the enterotype of the user is “a” among “a” to “e”, and the characteristics of the enterotype a are that “this type is the least among Japanese and includes relatively large amounts of OO bacteria and OO bacteria. It is recommended to ingest dark green vegetables and fish and shellfish.” On the screen in FIG. 9, “Your skin troubles” and “Dry skin” as the trouble contents are displayed. The user can grasp easily a user's own enterotype and user's own troubles only by visually recognizing the screen in FIG. 9 that is displayed on the user terminal 3.

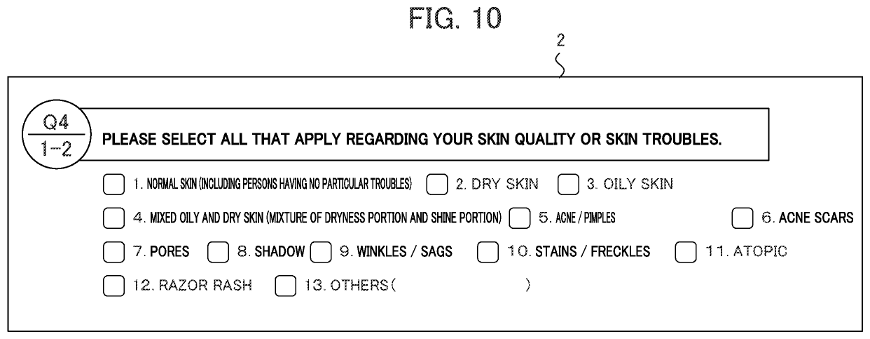

FIG. 10 is a diagram illustrating a specific example of the screen displayed on the user terminal when the user answers a questionnaire.

On the screen in FIG. 10, as one among a plurality of displayed questionnaires, “Please select all that apply regarding your skin quality or skin troubles.” is displayed and a plurality of choices are displayed so that the user can check the check boxes. Specifically, the choices are displayed which include “1. Normal skin (including persons having no particular troubles)”, “2. Dry skin”, “3. Oily skin”, “4. Mixed oily and dry skin (mixture of dryness portion and shine portion)”, “5. Acne/pimples”, “6. Acne scars”, “7. Pores”, “8. Shadow”, “9. Winkles/sags”, “10. Stains/freckles”, “11. Atopic”, “12. Razor rash”, and “13. Others”. The user can register a part of user's own information with the present service only by answering the questionnaire from the screen in FIG. 10 that is displayed on the user terminal 3.

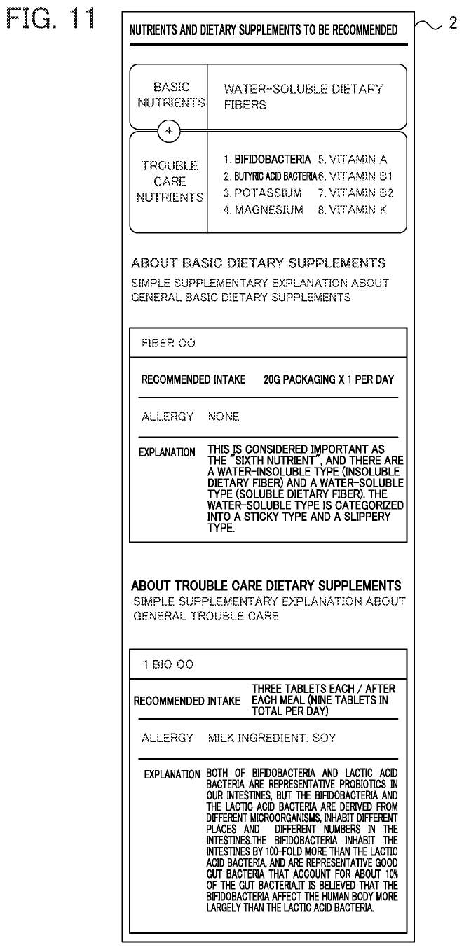

FIG. 11 is a diagram illustrating a specific example of the screen displayed on the user terminal when dietary supplements are recommended to the user.

On the screen in FIG. 11, “Nutrients and dietary supplements to be recommended” is displayed, and the basic nutrients (water soluble dietary fibers) and the trouble care nutrients (1. Bifidobacteria, 2. Butyric acid bacteria, 3. Potassium, 4. Magnesium, 5. Vitamin A, 6. Vitamin B1, 7. Vitamin B1, 8. Vitamin B1) are displayed.

In addition, “About basic dietary supplements” is displayed, and a simple supplementary explanation about general basic dietary supplements, and a product name and product information of a specific supplement to be recommended are displayed. Specifically, for example, the contents are displayed which include “Fiber OO”, “Recommended intake: 20 g packaging×1 per day”, “Allergy: None”, “Explanation: This is considered important as the “sixth nutrient”, and there are a water-insoluble type (insoluble dietary fiber) and a water-soluble type (soluble dietary fiber). The water-soluble type is categorized into a sticky type and a slippery type.”

In addition, “About trouble care dietary supplements” is displayed, and a simple supplementary explanation about general trouble care dietary supplements, and a product name and product information of a specific supplement to be recommended are displayed. Specifically, for example, the contents are displayed which include “Bio OO”, “Recommended intake: three tablets each/after each meal (nine tablets in total per day)”, “Allergy: Milk ingredient, Soy”, “Explanation: both of Bifidobacteria and lactic acid bacteria are representative probiotics in our intestines, but the Bifidobacteria and the lactic acid bacteria are derived from different microorganisms, inhabit different places and different numbers in the intestines. The Bifidobacteria inhabit the intestines by 100-fold more than the lactic acid bacteria, and are representative good gut bacteria that account for about 10% of the gut bacteria. It is believed that the Bifidobacteria affect the human body more largely than the lactic acid bacteria.” The user can acquire easily the information about the recommended dietary supplements only by visually recognizing the screen in FIG. 11 that is displayed on the user terminal 3.

In this way, in the present service, the recommendations are performed for the user on the basis of the gut dysbiosis score, the enterotype, and the data on the health of the user. This enables more appropriate suggestions to the user. Hereinafter, the reasons will be described.

That is, with the advancement of gut bacteria measuring technique, it is becoming possible to comprehensively measure and evaluate the total population beyond the method of measuring individual bacteria one by one. There is a concept of the gut flora (gut bacteria as the population) based on data obtained by such measurement and evaluation, as the notion that the gut bacteria are regarded as one ecosystem, for example. It has been conventionally understood that individual bacteria characters such as probiotics and bad bacteria are present and function beneficially or detrimentally. However, how the individual bacteria characters affect the total population of the gut flora is another subject.

First, in the present service, as the gut dysbiosis score, it can be determined that the gut dysbiosis score is in the “bad” condition. Specifically, for example, it can be determined that the normal balance of the gut flora has collapsed (referred to as the breakage of the intestinal tract barrier function, or Dysbiosis), and that the function is deteriorating. Here, such a state of collapse of the gut flora is found from a large number of patients due to various diseases. That is, it is believed that the state in which beneficial bacteria are reduced, the diversity decreases, specific bacteria proliferate abnormally, or the like refers to the state of collapse of the gut flora. In the present service, such a state is determined as the gut dysbiosis score being in the “bad” condition, on the basis of the stool specimen and the data on the health condition.

On the other hand, it is (relatively) difficult to determine that the gut dysbiosis score is in the “good” condition. That is, it can be determined that the gut flora is stable without collapsing, but it is difficult to determine the level of the “good” condition. Specifically, in the case of healthy persons, it is difficult to find common points on the gut flora due to a large difference between individuals. This may be because all of healthy persons have been exposed to different bacteria over the period from birth up to the present time. The growth environment and the dietary life when the persons were children are different individually, and therefore, it is believed that this leads to a difference in the gut flora between individuals. That is, it is not perfectly clear what enterotype is established. However, it is considered that one reason is that various kinds of gut bacteria have respective functions and function while cooperating or competing with each other.

In the present service, even in the users in the state in which the gut dysbiosis score is “good” (not bad), the suggestions are provided to the users using the enterotypes by focusing on classifying into some patterns (multiple-stability). In the present service, as a result of analysis of the gut bacteria data for the n subjects, the n subjects can be classified by type ( . . . enterotype) of the gut flora on the basis of the found enterotypes. It can be said that the present service focuses on the fact that foods, nutrients, and the like which are believed to be recommended for the user vary depending on the enterotype of the user.

In the present service, the gut flora of the user is determined on the basis of the gut dysbiosis score (good or bad) and the enterotype (what type), and the user is classified, and then the suggestions are provided. This enables the dietary supplements for regulating the entire gut flora to be recommended for the user in which the gut dysbiosis score approaches the bad condition. Regarding to the enterotype, the dietary supplements can be constituted on the basis of the information of nutrients which are believed to be recommended, depending on the enterotype, and can be incorporated in the recommendations.

The relatively general concept is that the gut dysbiosis score is described in the concept of “collapse of diversity” and dysbiosis as bad examples in the historical gut microbiota research. Specifically, since it is almost certain that the dysbiosis is associated with any disease or bad physical condition, many improved solutions (for example, intake of the lactic acid bacteria) have already been suggested. The measures against dysbiosis (so-called measures for “regulating the intestinal function” such as lactic acid bacteria) can be said to be established as industries, but the recommendations have not been provided for the users which are not applied to dysbiosis. That is, it is said that the measures against dysbiosis and the recommendations are separated.

The difference in the enterotype is a phenomenon which appears regardless of the deterioration of the gut microbiome. Then, in the present service, the difference in the enterotype further focuses on the fact that the persons with good gut dysbiosis score (that is, “healthy persons” in the gut microbiota) can be categorized into each type. In the present service, since “good or bad” of the gut dysbiosis score and the enterotype are conceptually distinguished, more appropriate suggestions can be provided for the user.

In this way, in the present service, for example, even in the “case of the type of the gut microbiota classified into a particular “type a”, “the gut flora may be in the good condition or the bad condition in the terms of “good or bad”, the “good or bad” and the type are determined distinguishably, and the recommendations can be provided using a combination of the “good or bad” and the “type”. This enables more appropriate suggestions to the user.

Although one embodiment of the present invention has been described above, the present invention is not limited to the above-described embodiment, and includes modifications, improvements, and the like within the scope which can achieve the object of the present invention.

For example, in the above-described embodiment, the scoring is performed using A, B, and C as the indicators of the gut dysbiosis score, but this is merely an illustrative example. Alternatively, as the gut dysbiosis score, the scoring may be performed using indicators other than the above-described A, B, and C.

For example, in the above-described embodiment, the types of “a” to “e” are used as the enterotype, but this is merely an illustrative example. Alternatively, as the enterotype, the types other than the above-described “a” to “e” may be used.

For example, in the above-described embodiment, as the recommendation processing, the dietary supplements are recommended for the user, but the present invention is not limited thereto. In the present service, in addition to the recommendations of the dietary supplements, the suggestions on nutrients, foods, medications, and physical exercise method may be provided.

For example, in the above-described embodiment, the gut bacteria data and the data on the health condition of each of the n subjects and the users are acquired, but the present invention is not limited thereto. That is, for example, it is only required that the gut bacteria data and the data on the health condition of each of the n subjects and the users are acquired. In this case, for example, the first management unit 115 stores in the suggestion DB 184 and manages suggestions by doctor or the like browsing the classification results, as suggestions for improving the health condition of persons belonging to the same group, for each of the groups classified by the classification unit 114. This enables the service provider to provide the above-described service. On the other hand, in the case where the gut bacteria data and the data on the health condition of each of the n subjects and the users are acquired, the first management unit 115 stores in the suggestion DB 184 and manages suggestions for improving the health condition of persons belonging to the same group, the suggestions being derived as the analysis results of the data on the health condition for each group.

For example, in the above-described embodiment, the data on the health condition is used for the grouping processing, the recommendation processing, and generation of the contents (suggestions) of the recommendations, but the present invention is not limited thereto. That is, for example, it is only required that at least the above-described enterotype and gut dysbiosis score are used for the grouping processing and the recommendation processing. As described above, the grouping processing and the recommendation processing are performed on the basis of the enterotype and the gut dysbiosis score, which enables more accurate grouping and recommendation as compared with the conventional technique. However, in addition to the enterotype, the gut dysbiosis score, and the information on the health, the other information may be used for the grouping processing and the recommendation processing.

For example, the configuration of the screen of the user terminal 3 in FIGS. 8 to 11 is merely an illustrative example, and therefore, a configuration other than the configuration of the screen illustrated in FIGS. 8 to 11 may be adopted.

The system configuration illustrated in FIG. 4 and the hardware configuration of the server 1 illustrated in FIG. 5 are merely an example for achieving the object of the present invention, and the present invention is not particularly limited to the example.

The functional block diagram in FIG. 6 is merely an illustrative example, and the present invention is not particularly limited to the example. That is, it is only required that the function capable of executing the above-described recommendation processing as a whole is provided in the information processing system in FIG. 4, and a functional block and database used to achieve this function are not particularly limited to the example of FIG. 6.

Furthermore, the locations of the functional block and database are not particularly limited to those in FIG. 6, but may be arbitrary. In the example in FIG. 6, a configuration is adopted in which the recommendation processing is performed by the control of the CPU 11 in the server 1 constituting the information processing system in FIG. 4, but is not limited thereto. For example, a configuration may be adopted in which at least a part of the functional block and database disposed on the server 1 side is provided on the user terminal 3 side or in the other information processing device (not illustrated).

The above-described series of processing can be executed by hardware or can be executed by software. In addition, one functional block may be configured with hardware alone, software alone, or a combination thereof.

When a series of processing is executed by software, a program included in the software is installed on a computer or the like from a network or a recording medium. The computer may be a computer which is incorporated in dedicated hardware. Alternatively, the computer may be a computer capable of executing various types of functions by installing various types of programs, such as a general-purpose smartphone or personal computer other than a server.

The recording medium having such programs includes a removable medium (not illustrated) distributed separately from a device main body to provide the programs to the user, and includes a recording medium or the like provided to the user in a state of being previously incorporated in the device main body.

In this specification, steps of describing the programs to be recorded on the recording medium include not only, as a matter of course, processing performed in time series according to its order but also processing executed in parallel or individually even when the processing is not necessarily in time sequence.

In summary, the information processing device to which the present invention is applied is sufficient if the present invention has the following configuration, and various embodiments may be taken. That is, it is only required that the information processing device to which the present invention is applied comprises:
acquisition unit (for example, a second acquisition unit 121 in FIG. 6) that acquires gut bacteria data of a user;
first determination unit (for example, a score determination unit 122 in FIG. 6) that determines a score (for example, a gut dysbiosis score in the specification) that indicates a health level of a gut microbiome of the user on a basis of the gut bacteria data;
second determination unit (for example, a type determination unit 123 in FIG. 6) that determines a type (for example, an enterotype in the specification) to which gut microbiota of the user applies, among a plurality of enterotypes on the basis of the gut bacteria data; and
information unit (for example, an information unit 125 in FIG. 6) that informs suggestions for improving a health condition of the user to the user on the basis of the score and the type of the user.

This makes it possible to provide suggestions tailored to the user on the basis of the health level of the gut microbiome of the user and the enterotype of the user. That is, more accurate suggestions (for example, suggestions on nutrients, foods, medications, and physical exercise method, and dietary supplements) for improving both health and beauty conditions of the users are enabled.

Furthermore, the information unit
can suggest intake of the dietary supplements (for example, a basic supplement and a trouble care supplement in FIG. 3) as the suggestions for improving the health condition of the user.

This makes it possible to suggest appropriate dietary supplements tailored to the health level of the gut microbiome of the user and the enterotype of the user.

Furthermore, the information unit
can inform at least one feature of the score and the type together with the suggestions for improving the health condition of the user and.

This enables the user to grasp user's own score and type and have daily life (for example, daily life in which foods and lifestyle are regulated) in consideration of the user' own score and type.

The information processing device further comprises:
in a state in which data including at least information on gut bacteria of each of n (n is an integer value of two or more) subjects is previously acquired as gut bacteria data (for example, a state in which gut bacteria data is acquired by a first acquisition unit 111 in FIG. 6),
first determination unit (for example, a score determination unit 112 in FIG. 6) that determines a score that indicates a health level of a gut microbiome of each of the n subjects on a basis of the gut bacteria data;
second determination unit (for example, a type determination unit 113 in FIG. 6) that determines a type to which gut microbiota of each of the n subjects applies, among a plurality of enterotypes on the basis of the gut bacteria data;
classification unit (for example, a classification unit 114 in FIG. 6) that classifies each of the n subjects into any one of a plurality of groups on the basis of the score and the type of each of the n subjects; and
management unit (for example, a first management unit 115 in FIG. 6) that manages suggestions for improving a health conditions of persons belonging to the same group for each of the groups,
wherein the information unit can inform suggestions, preset for a group to which the user belongs, to the user on a basis of the score and the type of the user.

That is, a score that indicates the health level of the gut microbiome of each of the n subjects is determined on the basis of the gut bacteria data, in the state in which the gut bacteria data of each of the n subjects is acquired. The type to which the gut microbiota of each of the n subjects applies is determined among a plurality of enterotypes on the basis of the gut bacteria data. Then, each of the n subjects is classified into any group on the basis of the score that indicates the health level of the gut microbiome of each of the n subjects and enterotype. Then, the suggestions for improving the health conditions of the users belonging to the group, the suggestions being preset for each group, are informed to the user.

Specifically, in the above-described embodiment, a gut dysbiosis score of each of the n subjects is determined in the basis of the gut dysbiosis score in the state in which the gut bacteria data of each of the n subjects is previously acquired. An enterotype of each of the n subjects is determined on the basis of the gut bacteria data. Then, each of the n subjects is classified into any one of a plurality of groups on the basis of the gut dysbiosis score and the enterotype of each of the n subjects. Then, the suggestions for improving the health conditions of the user belonging to the group, the suggestions being preset for each group, are informed to the user. This enables more accurate suggestions for improving both health and beauty conditions of the user.

The information unit
can suggest intake of the same kinds of dietary supplements (for example basic dietary supplements and trouble care dietary supplements in FIG. 3) as the suggestions for improving the health condition of one or more subjects belonging to the same group.

That is, intake of the same kinds of dietary supplements is suggested as suggestions for improving the health condition of one or more subjects belonging to the same group. In the above-described embodiment, intake of the same kinds of basic supplement and a trouble care supplement is suggested as the suggestions for improving the health condition of the one or more subjects belonging to the same group. This enables more accurate suggestions on dietary supplements for improving both health and beauty conditions of a subject.

### EXPLANATION OF REFERENCE NUMERALS

1. Server, 2. Subject terminal, 11. CPU, 12. ROM, 13. RAM, 14. Bus, 15. Input/output interface, 16. Input unit, 17. Output unit, 18. Storage unit, 19. Communication unit, 20. Drive, 40. Removable medium, 110. Grouping unit, 111. First acquisition unit, 112. Score determination unit, 1133. Type determination unit, 114. Classification unit, 115. First management unit, 120. Recommendation unit, 121. Second acquisition unit, 121. Score determination unit, 122. Type determination unit, 123. Second management unit, 124 Information unit, 181, Subject DB, 182. Classification DB, 183. Suggestion DB, NW. Network

1. An information processing device, comprising:
acquisition unit that acquires gut bacteria data of a user;
first determination unit that determines a score that indicates a health level of a gut microbiome of the user on a basis of the gut bacteria data;
second determination unit that determines a type to which gut microbiota of the user applies, among a plurality of enterotypes on the basis of the gut bacteria data; and
information unit that informs suggestions for improving a health condition of the user to the user on the basis of the score and the type of the user.

2. The information processing device according to claim 1, wherein
the information unit
informs suggestions on intake of dietary supplements as the suggestions for improving the health condition of the user.

3. The information processing device according to claim 1, wherein
the information unit
informs at least one feature of the score and the type together with the suggestions for improving the health condition of the user.

4. An information processing method, comprising:
an acquisition step of acquiring gut bacteria data of a user;
a first determination step of determining a score that indicates a health level of a gut microbiome of the user on a basis of the gut bacteria data;
a second determination step of determining a type to which gut microbiota of the user applies, among a plurality of enterotypes on the basis of the gut bacteria data; and
an information step of informing suggestions for improving a health condition of the user to the user on the basis of the score and the type of the user.

5. A non-transitory computer readable medium storing a program causing a computer to execute:
an acquisition step of acquiring gut bacteria data of a user;
a first determination step of determining a score that indicates a health level of a gut microbiome of the user on a basis of the gut bacteria data;
a second determination step of determining a type to which gut microbiota of the user applies, among a plurality of enterotypes on the basis of the gut bacteria data; and
an information step of informing suggestions for improving a health condition of the user to the user on the basis of the score and the type of the user.
